(19) United States
(12) Patent Application Publication
(10) Pub. No.: US 2018/0140227 A1
(43) Pub. Date: May 24, 2018

(54) METHOD AND APPARATUS FOR THE DETERMINATION OF BODY LUMEN DIMENSIONS

(71) Applicant: CSA Medical, Inc., Lexington, MA (US)

(72) Inventors: John P. O'Connor, Andover, MA (US); Brian M. Hanley, Reading, MA (US)

(21) Appl. No.: 15/814,900

(22) Filed: Nov. 16, 2017

Related U.S. Application Data
(60) Provisional application No. 62/424,147, filed on Nov. 18, 2016.

(51) Int. Cl. A61B 5/107; A61B 5/00; A61M 25/10

(52) U.S. Cl. CPC A61B 5/1076; A61B 8/12; A61M 25/10187; A61B 5/6853

(57) ABSTRACT

In various embodiments, the present disclosure pertains to methods for measuring a size of a body lumen. A method may comprise: (a) inserting a balloon comprising a circumference, a proximal end, and a distal end into the body lumen, wherein the balloon is at least partially compliant; (b) introducing an inflation fluid into the balloon inside the body lumen until a point in time where the circumference of the balloon engages an inner wall of the body lumen; (c) measuring an amount of the inflation fluid introduced into the balloon; and (d) determining the size of the body lumen based at least in part upon a total amount of inflation fluid introduced into the balloon at a point in time. In various embodiments, the present disclosure pertains to apparatuses for measuring an inner diameter of a body lumen.

### PRIORITY

This application dams the benefit of priority under 35 U.S.C. § 119 to U.S. Provisional Application Ser. No. 62/424,147, filed Nov. 18, 2016, which is incorporated by reference herein in its entirety and for all purposes.

### FIELD

The present disclosure pertains to methods and devices useful for determining the sizes of body lumens and for treating body lumens.

### BACKGROUND

When working inside a lumen in the body, for example, during the course of an endoscopic treatment, it is sometimes important to be able to determine dimensions of the body lumen for diagnostic and therapeutic approaches to be effective.

The present disclosure pertains to improved methods and devices useful for determining the sizes of body lumens and for treating body lumens.

### SUMMARY OF THE INVENTION

In various embodiments, the present disclosure pertains to methods for measuring a size of a body lumen in a subject. A method may include inserting a balloon having a circumference, a proximal end, and a distal end into the body lumen. A balloon may be at least partially compliant. A method may include introducing an inflation fluid into the balloon inside the body lumen until a point in time where the circumference of the balloon engages an inner wall of the body lumen. This may be around the entire perimeter of the inner wall. A method may include measuring an amount of the inflation fluid introduced into the balloon. A method may include determining the size of the body lumen based at least in part upon a total amount of inflation fluid introduced into the balloon at the point in time where the circumference of the balloon engages the inner wall of the body lumen. The inflation fluid may be a gas. A method may include measuring a pressure of the inflation fluid within the balloon.

In various embodiments, a total volume of the inflation fluid introduced into the balloon up until the point in time where the circumference of the balloon engages the inner wall of the body lumen may be measured or calculated and the circumference or diameter of the balloon may be calculated based on a geometry of the balloon. In some embodiments, a balloon may have a cylindrical or spheroidal shape. In some embodiments, an aspect ratio of the balloon, defined as L:D, where L is the axial length of the balloon and D is the maximum diameter of the balloon taken normal to the axial length, may range from 1:1 to 1:10. In some embodiments, the inflation fluid may be a gas and a plurality of measurements may be made, which may include (a) a measurement of the mass or volume of the inflation fluid introduced into the balloon, (b) a measurement of the pressure of the inflation fluid introduced into the balloon, and (c) optionally, a measurement of the temperature of the inflation fluid. The size of the body lumen may be determined based upon the plurality of measurements. In some embodiments, the size of the body lumen may be determined based on experimental data generated by inflation of the balloon in a series of tubes having known lumen sizes. For example, the size of the body lumen may be determined based on look-up tables, among other possibilities. In some embodiments, a pressure in the balloon at the point in time where the circumference of the balloon engages an inner wall of the body lumen is such that, if unconstrained, the circumference of the balloon may be at least 5% greater than a circumference of the inner wall of the body lumen being measured. The balloon may be inflated until characteristics of a curve of internal balloon pressure versus an amount of inflation fluid introduced is indicative as to whether the circumference of the balloon has engaged the inner wall of the body lumen. A method may further include treating the body lumen, wherein treating the body lumen is dictated by a determination that is made regarding the size of the body lumen. A method may further include treating the body lumen by spraying a cryogenic fluid from a catheter onto the surface of the body lumen, wherein the amount of cryogenic fluid that is sprayed from the catheter is dictated by a determination that is made regarding the size of the body lumen.

In various embodiments, the present disclosure pertains to apparatuses for measuring an inner diameter of a body lumen. An apparatus may be a catheter. The catheter may have a catheter lumen with a proximal end and a distal end. A balloon may have an internal volume, a proximal end, and a distal end. At least a portion of the balloon may be compliant. A proximal end of a baboon may be in fluid communication with a distal end of the catheter lumen. A source of inflation fluid may be in fluid communication with the proximal end of the catheter lumen. A flow sensor may be disposed between the source of inflation fluid and the balloon. A pressure sensor may be configured to measure a pressure exerted by the inflation fluid in the internal volume of the balloon. The inflation fluid may be a gas. A balloon may be formed from an elastomeric material selected from the group consisting of a thermoset elastomer and a thermoplastic elastomer. A balloon may have a cylindrical shape or a spheroidal shape. In some embodiments. An aspect ratio of a balloon, defined as L:D, where L is the axial length of the balloon and D is the maximum diameter of the balloon taken normal to the axial length, may range from 1:1 to 1:10.

In various embodiments, a balloon may have a cylindrical shape including a circular proximal end wall, a circular distal end wall, and a side wall that connects and separates the proximal and distal end walls. In some embodiments, a material forming the circular proximal end wall and circular distal end wall of the balloon may stretch isotropically. In some embodiments, a material forming a side wall of the baboon may stretch anisotropically in a direction aligned with a circumference of the side wall.

These and other aspects and embodiments of the present disclosure, will have various benefits and advantages associated with the present disclosure, which will become immediately apparent to those of ordinary skill in the art upon reading the Detailed Description to follow.

### DRAWINGS

FIG. 1A is a schematic perspective view of a balloon for use in a catheter-balloon assembly, in accordance with an embodiment of the present disclosure.

FIG. 1B is a schematic side view of the balloon of FIG. 1.

FIG. 1C is a schematic top view of the balloon of FIG. 1.

FIG. 2 is a schematic illustration of an apparatus for body lumen measurement, in accordance with an embodiment of the present disclosure.

FIG. 3 is a schematic illustration of a procedure in which the apparatus shown in FIG. 2 and an endoscope are used in a procedure, in accordance with an embodiment of the present disclosure.

FIG. 4 is a schematic cross-sectional view of a collapsed, folded balloon for use in a catheter-balloon assembly, in accordance with an embodiment of the present disclosure.

### DETAILED DESCRIPTION

In some embodiments of the present disclosure, an apparatus, including a catheter-balloon assembly, which will be described in more detail below, is used to measure one or more body lumen dimensions using a process that comprises positioning a balloon at a target location within the body lumen (e.g., a treatment location), expanding the balloon until it engages an inner wall of the body lumen, and calculating one or more body lumen dimensions at the target location. In general, when a balloon is “engaged” with an inner wall of a body lumen, an appropriate region of the balloon is in contact with an entire perimeter of the inner wall of the body lumen.

Expanding the balloon includes inflating the balloon with a fluid. The inflation fluid may be a compressible fluid (e.g., a suitable gas such as air, nitrogen, oxygen, etc.) or a substantially incompressible fluid (e.g., a suitable liquid such as water, normal saline, etc.). The inflation fluid may be provided from any suitable fluid source. For example, the fluid source may comprise a pressurized source of gas such as a pressurized cylinder or an air compressor, among other possibilities, or the fluid source may comprise a pressurized source of a liquid, such as a positive displacement pump, among other possibilities.

In various embodiments, the body lumen dimensions are calculated based on the amount of the inflation fluid introduced to the balloon during the course of balloon inflation. For example, a suitable flow sensor may be employed to sense flow of compressible or substantially incompressible fluid from the fluid source into the balloon. For instance, the flow sensor may comprise a mass flow sensor or a volume flow sensor.

Where the inflation fluid is a liquid, the inflation fluid may be assumed to be incompressible in some embodiments. Consequently, where the mass of the inflation fluid is known (e.g., measured using a mass flow sensor), the volume V (m³ in SI units) can be calculated based on the mass of the fluid m (kg in SI units) and the density of the fluid ρ (kg/m³ in SI units) using the equation V=mρ. The density at body temperature may be used for calculations, as liquids are typically relatively constant over temperature.

Where the inflation fluid is a gas, the volume will be dependent upon the quantity of gas introduced into the balloon as well as the pressure and temperature of the gas. In this regard, the ideal gas law predicts that the volume of a gas V (m³ in SI units) can be calculated based on the gas constant R (8.31441 J K⁻¹ mol⁻¹ in SI units), the pressure of the gas P (Pa, in SI units), the temperature T of the gas (degrees kelvin in SI units), and the number of moles, n, of the gas, according to the relationship V=nRT/P. Where the mass of the gas, m (kg in SI units), is measured, for example, using a mass flow sensor, the relationship can be expressed as V=mRT/(MP), where M is the molecular weight of the gas (kg/mol).

In various embodiments, a suitable pressure sensor may be provided for determining the internal pressure in the balloon arising from the introduction of the inflation fluid. A pressure sensor may be placed, for example, inside the balloon or placed in fluid communication with a lumen that supplies inflation fluid to the balloon. The pressure sensor may comprise any device suitable for determining pressure inside an enclosed space including a strain gauge such a piezoresistive strain gauge, capacitive strain gauge, electromagnetic strain gauge, optical strain gauge or potentiometric strain gauge, among others. The pressure sensor may be used to determine when the balloon has engaged a surrounding body lumen. Moreover, where the fluid is a compressible fluid such as a gas, the volume of fluid in the baboon may be calculated based on the measured pressure, the measured mass of inflation fluid that is introduced to the balloon and the measured (or assumed) temperature.

A temperature sensor may also be optionally provided in some embodiments to determine the internal temperature of the fluid in the balloon. In other embodiments, the temperature may be assumed to be body temperature. In this regard, where the inflation fluid is a liquid, the density of the fluid is relative constant with temperature. For instance, the density of water at room temperature (298 K) is 997.13 kg/m³, while the density of water at body temperature (310 K) is 993.37 kg/m³. Where the fluid is a gas, body temperature may also be assumed in some embodiments, as small differences in temperature are not particularly consequential as a result of the Kelvin temperature scale, in which room temperature (298 K) differs from body temperature (310 K) by only about 4%. Moreover, the deviation is likely to be less than this value, because the temperature inside the balloon is typically much closer to, or virtually the same as, body temperature, when the balloon is positioned in a living subject.

The balloon may be selected from any of a variety of shapes including that of a spheroid (i.e., a sphere, oblate spheroid or prolate spheroid) or a cylinder. Where the balloon is in the shape of a cylinder, the relationship between balloon volume, V, balloon diameter, D, and balloon length, L, is V=π(D/2)²L, and V can be used to calculate the D, assuming L is known. Where the balloon is in the shape of a spheroid, whether a sphere, an oblate spheroid or prolate spheroid, the relationship between volume, V, equatorial diameter, D (the diameter of interest), and polar diameter, C, is V=π/6D²C. Since C=D for a sphere, V=π/6D³.

The balloon may have any of a variety of aspect ratios, A, defined herein as A=L:D, where L is the axial length of the shape (for a spheroid, equal to C) and D the diameter of the shape. For example, for a cylinder, A may range from 5:1 or more to 1:10 or less, for example, ranging from 5:1 to 2:1 to 1:1 to 1:2 to 1:5 to 1:10, typically ranging from 1:1 to 1:10. Similarly, for a spheroid, A may range from 5:1 or more to 1:10 or less, for example, ranging from 5:1 (prolate spheroid) to 2:1 (prolate spheroid) to 1:1 (sphere) to 1:2 (oblate spheroid) to 1:5 (oblate spheroid) to 1:10 (oblate spheroid), typically ranging from 1:1 to 1:10.

Once the volume of the fluid in the balloon is determined, the diameter of the balloon can be calculated based on the geometry of the balloon that is employed, for example, using the equations set forth above, among others. The diameter of the lumen within which the baboon has been inflated can be presumed to have a diameter equal to that of the balloon. In some embodiments, the balloon diameter may be determined based on lookup tables that have been generated based on experimental testing.

The baboon may be formed of any suitable thin, compliant or non-compliant flexible material. In various embodiments, the balloon may be formed using a compliant polymeric material or a non-compliant polymeric material.

Examples of compliant polymeric materials include thermoset elastomers such as polysiloxanes (e.g., silicone rubber), polyisoprene, styrene butadiene rubber, ethylene propylene diene monomer rubber, polychloroprene rubber, chlorosulfonyl polyethylene rubber, acrylonitrile butadiene rubber, polyacryclic rubber, ethylene acrylic rubber, epichlorohydrin rubber, polyisobutylene rubber, hydrogenated nitrile rubber, fluorocarbon rubber, fluorosilicone rubber, and perfluorocarbon rubber, among other materials. Examples of compliant polymeric materials also include thermoplastic elastomers such as thermoplastic urethanes including thermoplastic polyester urethanes and thermoplastic polyether urethanes, styrenic block copolymers including styrene-butadiene-styrene block copolymer, styrene-isoprene-styrene block copolymer, styrene-ethylene-butadiene-styrene block copolymer, styrene-ethylene-propylene-styrene block copolymer, thermoplastic poly(ether ester) elastomers, and polyester amide) elastomers, among other materials. For compliant polymeric materials, the durometer range of the polymer may range, for example, from Shore A 30 to Shore A 80, among other possibilities.

Examples of non-compliant polymeric materials include polyethylene terephthalate (PET) and nylon family materials such as Grilamid® and Vestamid®, among other materials.

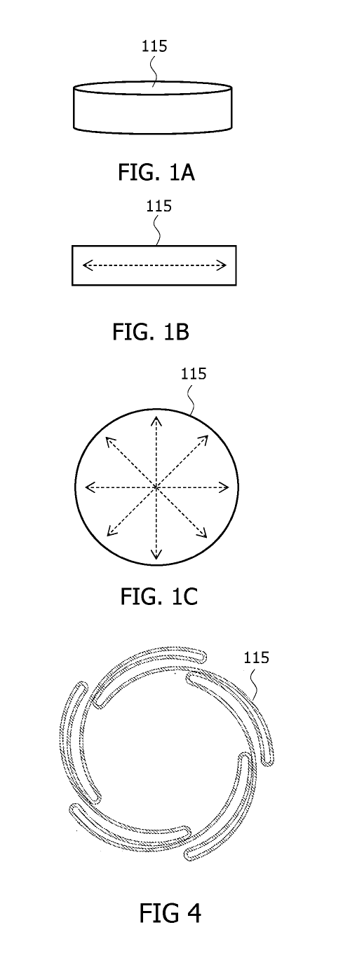

In certain embodiments, the baboon may be formed from an isotropically expandable material. In certain embodiments, the balloon may be formed from an anisotropically expandable material. In certain embodiments, the balloon may be formed from a combination of isotropically and anisotropically expandable materials, an example of which is illustrated in FIGS. 1A-1C. In this regard, FIG. 1A is a schematic perspective view of a cylindrical balloon 115 for use in conjunction with the present disclosure having an aspect ratio of 1:3. In a beneficial embodiment, the circular ends of the cylinder are isotropically expandable equally expandable in all directions) as schematically illustrated by the dashed arrows in FIG. 1C, whereas the side of the cylinder is anisotropically expandable as schematically illustrated by the dashed arrow in FIG. 1B such that the expansion occurs predominantly in the diameter of the cylinder and expansion in the length of the cylinder is minimal. In this case V=π(D/2)²L, where L is assumed to be constant.

The diameter of the balloon that is employed will depend on the body lumen that is being measured and will be greater than the diameter of the body lumen being measured when inflated to measurement pressure, typically from 5% to 25% greater than the anticipated body lumen diameter. As used herein “measurement pressure” is the pressure within the balloon at the point in time where the balloon is properly engaged with the inner wall of the body lumen as discussed in more detail below and may be based, for example, on a preset pressure value or on an analysis of changes in pressure that are observed upon the introduction of inflation into the balloon while in the body. For example, where the body lumen is an adult human esophagus, the balloon diameter at measurement pressure may range, for example, from 20 to 32 mm among other values; where the body lumen is an adult human colon, the balloon diameter at measurement pressure may range, for example, from 20 to 40 mm among other values; and where the body lumen is an adult stomach, the balloon diameter at measurement pressure may range, for example, from 20 to 100 mm among other values.

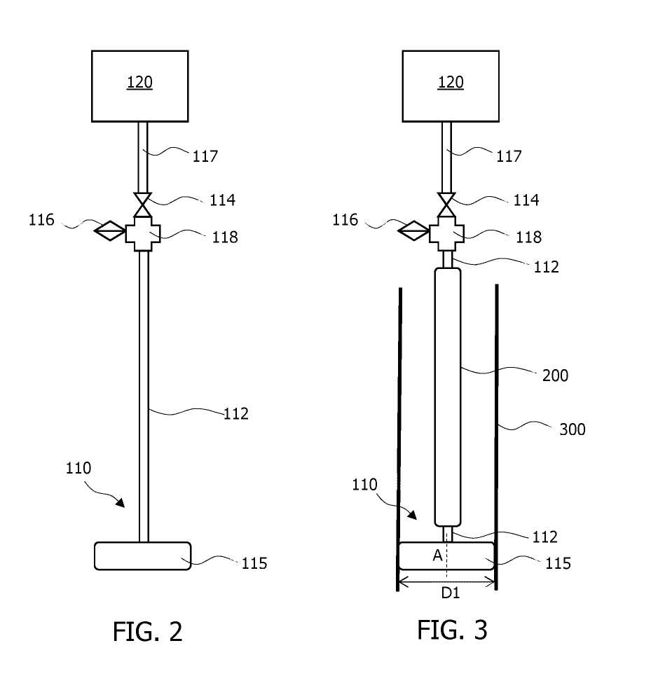

One particular embodiment of an apparatus in accordance with the present disclosure is shown in FIG. 2. The apparatus comprises a balloon 115 for determining the dimensions of a body lumen in fluid communication with a fluid source 120. Although shown in an expanded configuration, in use balloon 115 is inserted into a body lumen in a collapsed configuration and expanded upon proper placement at a pre-selected treatment area. For example, the balloon 115 may be provided in a collapsed and folded configuration having a cross-section analogous to that shown in FIG. 4. The balloon 115 is attached to a distal end of a catheter tube 112 for flowing an inflation fluid into the baboon 115 from the fluid source 120. (Combination of the balloon 115 and the catheter tube 112 are collectively referred to herein as a balloon-catheter assembly 110.)

The fluid source 120 may be connected to an access port 118 located at the proximal end of the catheter tube 112 via a length of tubing 117. Positioned between the source 130 and the balloon 115 is a flow sensor 114 for measuring the amount of fluid that that flows from the fluid source 120 to the balloon 115 as the balloon 115 is inflated. Also provided is a pressure sensor 116, which is connected to the access port 118 in the embodiment shown.

In one aspect of the disclosure, a method for measuring one or more internal dimensions at a location in a body lumen comprises positioning a balloon at a location within the body lumen, inflating the balloon with an inflation fluid to engage an inner wall of the body lumen, and determining the one or more internal dimensions of the lumen based, at least in part, on the amount of fluid that is introduced into the balloon.

In this regard, and with reference to FIG. 3, in various embodiments, using an apparatus like that of FIG. 2, the balloon 115 of the balloon-catheter assembly 110 is delivered to a location within a body lumen 300 by advancing the balloon 115 through the body lumen 300. In certain embodiments, the balloon-catheter assembly 110 may be delivered with the assistance of an additional device such as an endoscope 200 as shown in FIG. 3. The balloon 115 is typically in a collapsed configuration during the delivery to the target site in the body lumen 300. Examples of endoscopes which may be employed include the following, among others (listed along with the body lumen or lumens typically viewed with the same): bronchoscopes (trachea and bronchi of the lungs), colonoscopes (colon and large intestine), esophagoscopes (esophagus), gastroscopes (stomach including hiatal hernias, and duodenum), proctoscopes (rectum and sigmoid colon), sigmoidoscopes (sigmoid colon), and cystoscopes (bladder), among others.

Once delivered to the desired location, the balloon may be filled with an inflation fluid from the fluid source 120 until the balloon 115 engages the inner wall of the body lumen 300. As previously indicated, the balloon 115 is oversized so that the diameter of the balloon when unrestrained at measurement pressure is larger than the diameter of the baboon when constrained in the body lumen at measurement pressure. As the inflation fluid is injected into balloon 115, the balloon expands radially from its axis A (illustrated by a dashed one) to engage an inner wall of the body lumen 300. As previously noted, the balloon 115 may be filled with an inflation fluid that is an incompressible fluid such as normal saline or a compressible fluid such as air to a point where the balloon is engaged with the body lumen. During the introduction of the inflation fluid, the amount of inflation fluid being injected into the balloon may be measured using flow sensor 114, the pressure of the fluid within the balloon may be measured using pressure sensor 116 and, in some cases, the temperature of the fluid within the balloon is measured using a temperature sensor (not shown). Based on this information, a determination may be made regarding the dimension of the lumen 300. Subsequently, the balloon may be deflated so that it can be readily removed from the target site.

The point at which the balloon 115 is properly engaged with the inner wall of the lumen 300 may be determined in various ways. For example, in certain embodiments, proper engagement with the body lumen may be verified by visual observation.

In certain embodiments, proper engagement may be determined based on the internal pressure of the inflation fluid as measured by the pressure sensor 116. For example, proper engagement with the body lumen may be assumed upon the inflating the balloon 115 to a predetermined pressure (e.g., 1 atm=14.7 psig=101325 Pa) where it is known that the baboon, due to is size relative to the body lumen, will be in engagement with the body lumen.

As another example, for instance, where the balloon is formed of a material that is more compliant than the body lumen, proper engagement may be reflected by a significant increase in a slope of a curve of pressure in the balloon vs. quantity of inflation fluid introduced into the balloon (or a curve of pressure in the balloon vs. time, where a constant mass flow rate of inflation fluid is infused into the balloon). In this regard, and without wishing to be bound by theory, where a compliant balloon is employed, the pressure in the balloon may increase, for example, initially as a result of elastic forces exerted by the balloon and, once the balloon expands to engage the wall of a surrounding body lumen body lumen, further due to forces exerted on the balloon by the body lumen wall, which is expected to result in a change in the relationship between the pressure in the balloon and the incremental amounts of inflation fluid that are introduced.

As noted above, by measuring the amount of fluid that is introduced into the balloon, and in some cases the pressure and/or temperature of the fluid in the balloon, the volume of fluid in the balloon can be determined. Based on this information, the internal dimension of the body lumen may be calculated based on the geometry of the balloon.

Alternatively, the internal dimension of the body lumen may be calculated from pre-established correlations (e.g., using look-up tables, etc.) between (a) lumen diameter and (b) one or more of the following measurements, among others: (i) the measured volume of fluid introduced, (ii) the measured volume of fluid introduced, the measured pressure, and optionally, the measured temperature, (iii) the measured mass of fluid introduced, and (iii) the measured mass of fluid introduced, the measured pressure and, optionally, the measured temperature.

For example, ex vivo tests may be performed by inserting the balloon 115 into a series of tubes having different diameters (which may be rigid or may be formed of a material approximating the compliance of the body lumen being measured) and inflating the balloon 115 to a point where the balloon 115 is properly engaged with the inner wall of the lumen of the tubes, while measuring various quantities such as (a) volume or mass of fluid introduced, (b) pressure and (c) optionally, temperature. This data may be used to establish correlations such as those described above.

Similar tests may also be used, for example, to establish a suitable pre-determined pressure to which the balloon be inflated and engagement with a surrounding lumen (within a range of lumen sizes) can be safely assumed to have occurred, or to establish characteristics of a curve of pressure vs. quantity of inflation fluid introduced (or a curve of pressure vs. time where a constant mass flow rate of inflation fluid is infused into the balloon) that indicate that contact has been made with the surrounding lumen, among other uses.

In some embodiments, the target site is further subjected to a treatment step after lumen measurement, which treatment may depend upon the results of the lumen measurement. For example, in one specific embodiment a spray cryotherapy is to be performed in the body lumen 300 in which an ablation of benign or malignant tissue may be accomplished.

1. A method for measuring a size of a body lumen comprising:
inserting a balloon comprising a circumference, a proximal end, and a distal end into the body lumen, wherein the balloon is at least partially compliant;
introducing an inflation fluid into the balloon inside the body lumen until a point in time where the circumference of the balloon engages an inner wall of the body lumen;
measuring an amount of the inflation fluid introduced into the baboon; and
determining the size of the body lumen based at least in part upon a total amount of inflation fluid introduced into the balloon at said point in time.

2. The method of claim 1, wherein the inflation fluid is a gas.

3. The method of claim 1, further comprising measuring pressure of the inflation fluid within the balloon.

4. The method of claim 1, wherein a total volume of the inflation fluid introduced into the balloon at said point in time is measured or calculated and wherein the circumference or diameter of the balloon is calculated based on a geometry of the balloon.

5. The method of claim 4, wherein the balloon has a cylindrical or spheroidal shape.

6. The method of claim 5, wherein an aspect ratio of the balloon, defined as L:D, where L is an axial length of the balloon and D is a maximum diameter of the balloon taken normal to the axial length, ranges from 1:1 to 1:10.

7. The method of claim 1, wherein a pressure in the balloon at said point in time is such that, if unconstrained, the circumference of the balloon would be at least 5% greater than a circumference of the inner wall of the body lumen being measured.

8. The method of claim 1, wherein the balloon is inflated until characteristics of a curve of internal balloon pressure versus an amount of inflation fluid introduced is indicative as to whether the circumference of the balloon has engaged the inner wall of the body lumen.

9. The method of claim 1, further comprising treating the body lumen, wherein treating the body lumen is dictated by a determination that is made regarding the size of the body lumen.

10. The method of claim 1, further comprising treating the body lumen by spraying a cryogenic field from a catheter onto a surface of the body lumen, wherein an amount of cryogenic fluid that is sprayed from the catheter is dictated by a determination that is made regarding the size of the body lumen.

11. A method for measuring a size of a body lumen comprising:
inserting a balloon comprising a circumference, a proximal end, and a distal end into the body lumen, wherein the balloon is at least partially compliant;
introducing an inflation fluid into the balloon inside the body lumen until a point in time where the circumference of the balloon engages an inner wall of the body lumen;
wherein a plurality of measurements is made that comprise:
(a) a measurement of a mass or a volume of the inflation fluid introduced into the balloon;
(b) a measurement of a pressure of the inflation fluid introduced into the balloon; and
(c) optionally, a measurement of a temperature of the inflation fluid; and
wherein the size of the body lumen is determined based upon the plurality of measurements.

12. The method of claim 11, wherein the size of the body lumen is determined based on experimental data generated by inflation of the balloon in a series of tubes having known lumen sizes.

13. The method of claim 12, wherein the size of the body lumen is determined based on look-up tables.

14. An apparatus for measuring an inner diameter of a body lumen comprising:
a catheter having a catheter lumen with a proximal end and a distal end;
a balloon comprising an internal volume, a proximal end and a distal end, wherein at least a portion of the balloon is compliant and a proximal end of said balloon is in fluid communication with the distal end of the catheter lumen;
a source of inflation fluid in fluid communication with the proximal end of the catheter lumen;
a flow sensor disposed between the source of inflation fluid and the balloon; and
a pressure sensor configured to measure a pressure exerted by the inflation fluid in the internal volume of the balloon.

15. The apparatus of claim 14, wherein the inflation fluid is a gas.

16. The apparatus of claim 14, wherein the balloon is formed from an elastomeric material selected from the group consisting of a thermoset elastomer and a thermoplastic elastomer.

17. The apparatus of claim 14, wherein the balloon has a cylindrical shape or a spheroidal shape.

18. The apparatus of claim 14, wherein an aspect ratio of the balloon, defined as L:D, wherein L is an axial length of the balloon and D is an maximum diameter of the balloon taken normal to the axial length, ranges from 1:1 to 1:10.

19. The apparatus of claim 14, wherein the balloon has a cylindrical shape comprising:
a circular proximal end wall;
a circular distal end wall; and
a side wall that connects and separates the proximal and distal end walls,
wherein an aspect ratio of the balloon, defined as L:D, wherein D is a diameter of the circular proximal and distal end walls and L is a height of the side wall, ranges from 1:1 to 1:10.

20. The apparatus of claim 19, wherein a material forming the circular proximal and distal end walls of the balloon stretch isotropically and wherein the material forming the side wall of the balloon stretches anisotropically in a direction aligned with a circumference of the side wall.
